(19) United States
(12) Patent Application Publication
(10) Pub. No.: US 2026/0137722 A1
(43) Pub. Date: May 21, 2026

(54) METHOD FOR PREPARING EXOSOME AND USE OF EXOSOME PREPARED THEREBY

(71) Applicant: Chang Gung Memorial Hospital, Linkou, Taoyuan City (TW)

(72) Inventors: Hung-Chi Chen, Taoyuan City (TW); Yi-Jen Hsueh, Taoyuan City (TW)

(21) Appl. No.: 19/079,114

(22) Filed: Mar. 13, 2025

(30) Foreign Application Priority Data
Nov. 21, 2024 (TW) 113144900

(51) Int. Cl. A61K 35/30; A61K 9/50; A61P 27/02

(52) U.S. Cl. CPC A61K 35/30; A61K 9/5068; A61P 27/02

(57) ABSTRACT

A method for preparing an exosome includes: cultivating corneal stromal keratocytes (CSKs) in a culture medium containing vitamin C, so as to obtain a cell culture of the CSKs containing the exosome; and subjecting the cell culture to an isolation treatment, so as to obtain the exosome. An exosome prepared by the aforesaid method, and methods for promoting proliferation of corneal endothelial cells and improving wound healing in a corneal endothelium using the exosome are also provided.

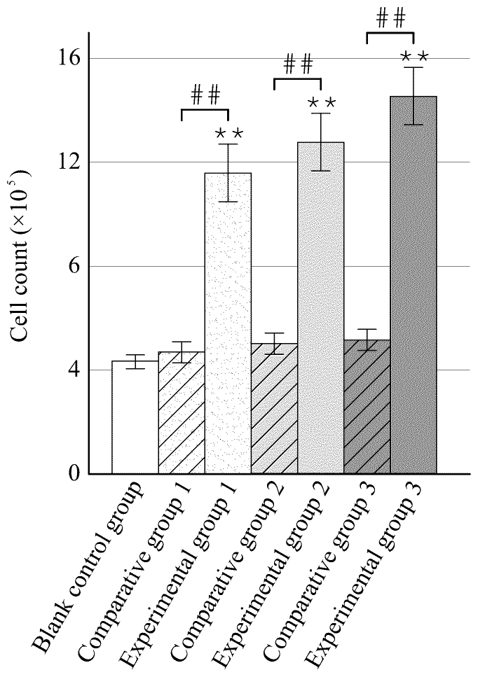

### CROSS-REFERENCE TO RELATED APPLICATION

This application claims priority to Taiwanese Invention patent application No. 113144900, filed on Nov. 21, 2024, the entire disclosure of which is incorporated by reference herein.

### FIELD

The present disclosure relates to a method for preparing an exosome. The disclosure also relates to an exosome prepared by the aforesaid method, and methods for promoting proliferation of corneal endothelial cells and improving wound healing in a corneal endothelium using the exosome.

### BACKGROUND

A corneal endothelium, constituting of corneal endothelial cells (CECs), is capable of maintaining corneal transparency by regulating water content of the cornea. However, these CECs lack regenerative ability, and a cell density of the corneal endothelium naturally declines with age. When the corneal endothelium is damaged, wound healing and repair are achieved through migration and enlargement of neighboring CECs. In cases of severe damage, endothelial keratoplasty is required for effective treatment.

In a previous study by Hsueh Y. J. et al. (2021), Biomed. Pharmacother., 144:112306, the applicant of this disclosure discovered that ascorbic acid (also known as vitamin C) is transported into CECs via glucose transporter 1 (GLUT1), such that the ascorbic acid increases extracellular signal-regulated kinase (ERK) phosphorylation and promotes cell proliferation.

In view of the aforesaid, there is still a need to develop an effective way for promoting proliferation of corneal endothelial cells, and hence improving wound healing in the corneal endothelium.

### SUMMARY

Therefore, in a first aspect, the present disclosure provides a method for preparing an exosome, which can alleviate at least one of the drawbacks of the prior art, and which includes: cultivating corneal stromal keratocytes (CSKs) in a culture medium containing vitamin C, so as to obtain a cell culture of the CSKs containing the exosome; and subjecting the cell culture to an isolation treatment, so as to obtain the exosome.

In a second aspect, the present disclosure provides an exosome, which can alleviate at least one of the drawbacks of the prior art. The exosome is prepared by the aforesaid method.

In a third aspect, the present disclosure provides a method for promoting proliferation of corneal endothelial cells, which can alleviate at least one of the drawbacks of the prior art, and which includes administering to a subject in need thereof a composition including the aforesaid exosome.

In a fourth aspect, the present disclosure provides a method for improving wound healing in a corneal endothelium, which can alleviate at least one of the drawbacks of the prior art, and which includes administering to a subject in need thereof a composition including the aforesaid exosome.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

The sole FIGURE shows the cell count determined in each group of Example 2, infra, in which the symbol “**” represents p<0.01 (compared with the blank control group), and the symbol “##” represents p<0.01 (compared between the experimental group 1 and the comparative group 1, the experimental group 2 and the comparative group 2, and the experimental group 3 and the comparative group 3).

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for preparing an exosome, which includes: cultivating corneal stromal keratocytes (CSKs) in a culture medium containing vitamin C, so as to obtain a cell culture of the CSKs containing the exosome; and subjecting the cell culture to an isolation treatment, so as to obtain the exosome.

As used herein, the term “corneal stromal keratocytes (CSKs)” can be used interchangeably with other terms such as “keratocytes” and “corneal fibroblasts”, and is intended to encompass the CSKs that are readily accessible to those skilled in the art (e.g., commercially available CSKs purchased from domestic or foreign depositaries) or that are isolated from a biological sample using techniques well-known to those skilled in the art (e.g., CSKs isolated from residual tissue samples after endothelial keratoplasty). In this regard, those skilled in the art may refer to relevant publications, e.g., Binte M Yusoff N. Z. et al. (2022), Cells, 5; 11 (1): 178.

According to the present disclosure, the CSKs in the culture medium containing the vitamin C may have an initial count ranging from 5×10⁶ cells to 1×10⁷ cells. In certain embodiments, the CSKs in the culture medium containing the vitamin C has an initial count of 8×10⁶ cells.

According to the present disclosure, the culture medium may contain the vitamin C which has a concentration ranging from 0.5 mM to 1.0 mM. In certain embodiments, the culture medium contains the vitamin C which has a concentration of 1.0 mM.

As used herein, the term “cultivating” can be used interchangeably with other terms such as “cultivation.” The procedures and conditions for cultivation may be adjusted according to practical requirements. In this regard, those skilled in the art may refer to relevant publications, e.g., Hsueh Y. J. et al. (2020), J. Cell Mol. Med., 24 (12): 6596-6608.

According to the present disclosure, cultivation may be conducted at a temperature of 37° C. for a time period ranging from 24 hours to 72 hours. In certain embodiments, cultivation is conducted at a temperature of 37° C. for a time period of 48 hours.

According to the present disclosure, the isolation treatment may be performed using techniques well-known to those skilled in the art. Examples of the isolation treatment may include, but are not limited to, a polymer-based precipitation treatment, a size-based separation treatment, and a density-based separation treatment. In this regard, those skilled in the art may refer to relevant publications, e.g., Welsh J. A. et al. (2024), J. Extracell. Vesicles., 13(2): e12404.

In certain embodiments, the polymer-based precipitation treatment may be selected from the group consisting of a polyethylene glycol (PEG) precipitation treatment, a dextran precipitation treatment, and a combination thereof.

In certain embodiments, the size-based separation treatment may be selected from the group consisting of a filtration treatment (e.g., an ultrafiltration treatment and a tangential flow filtration (TFF) treatment), a dialysis treatment (e.g., a diafiltration treatment), a chromatography treatment (e.g., a size-exclusion chromatography (SEC) treatment), and combinations thereof.

In certain embodiments, the density-based separation treatment may be a centrifugation treatment which is selected from the group consisting of an ultracentrifugation treatment and a density gradient centrifugation treatment.

In an exemplary embodiment, the exosome is obtained by subjecting the cell culture to the PEG precipitation treatment using an ExoQuick-TC kit.

The present disclosure also provides an exosome, which is prepared by the aforesaid method.

According to the present disclosure, the exosome may have a particle size ranging from 50 nm to 150 nm. In certain embodiments, the exosome may have a particle size ranging from 100 nm to 120 nm.

According to the present disclosure, the exosome may have a mean particle size ranging from 60 nm to 140 nm. In certain embodiments, the exosome has a mean particle size of 110 nm.

The present disclosure also provides a method for promoting proliferation of corneal endothelial cells, which includes administering to a subject in need thereof a composition including the aforesaid exosome.

As used herein, the term “administering” can be used interchangeably with other terms such as “administration,” and means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats.

In certain embodiments, the subjects may suffer from corneal endothelial damage. In other embodiments, the subjects may suffer from reduced density of corneal endothelial cells.

The present disclosure also provides a method for improving wound healing in a corneal endothelium, which includes administering to a subject in need thereof a composition including the aforesaid exosome.

As used herein, the term “wound healing” can be used interchangeably with other terms such as “wound repair.”

According to the present disclosure, the composition may be prepared in the form of a pharmaceutical composition. The pharmaceutical composition may be formulated into a dosage form suitable for intraocular or topical ophthalmic administration using technology well-known to those skilled in the art.

For intraocular administration, the pharmaceutical composition according to the present disclosure may be formulated into an injection, e.g., a sterile aqueous solution, a dispersion, or an emulsion.

The pharmaceutical composition according to the present disclosure may be administered via one of the following intraocular routes: subtenon injection, intravitreal injection, intracameral injection, intra-retinal injection, subretinal injection, and suprachoroidal injection.

According to the present disclosure, examples of the dosage form suitable for topical ophthalmic administration may include, but are not limited to, drops, emulsions, gels, ointments, creams, sprays, micelles, and suspensions.

According to the present disclosure, the pharmaceutical composition may further include a pharmaceutically acceptable carrier widely employed in the art of drug-manufacturing. For instance, the pharmaceutically acceptable carrier may include one or more of the following agents: solvents (e.g., sterile water), buffers (e.g., an ophthalmic balanced salt solution, phosphate-buffered saline (PBS), Ringer's solution, and Hank's solution), emulsifiers, suspending agents, decomposers, pH adjusting agents, stabilizing agents, chelating agents, preservatives, diluents, absorption delaying agents, liposomes, lubricants, and the like. The choice and amount of the aforesaid agents are within the expertise and routine skills of those skilled in the art.

According to the present disclosure, the dose and frequency of administration of the exosome may vary depending on the following factors: the severity of the illness or disorder to be treated, routes of administration, and age, physical condition and response of the subject to be treated. In general, the exosome may be administered in a single dose or in several doses. In certain embodiments, the exosome is administered in a single dose.

The disclosure will be further described by way of the following examples. However, it should be understood that the following examples are solely intended for the purpose of illustration and should not be construed as limiting the disclosure in practice.

### EXAMPLES

### Example 1. Preparation of Exosome Using Corneal Stromal Keratocytes (CSKs) Treated with Vitamin C

### Experimental Materials:

### 1. Source and Cultivation of CSKs

CSKs (purchased from Innoprot) were grown in a 10-cm Petri dish containing Dulbecco's modified Eagle medium/nutrient mixture F-12 (DMEM/F12) medium (Manufacturer: Gibco, Cat. no.: 11330-032) supplemented with 2% fetal bovine serum (FBS, Manufacturer: Gibco, Cat. no.: 10437-028), 1× insulin-transferrin-selenium (ITS, Manufacturer: Sigma-Aldrich, Cat. no.: 13146), 25 μg/mL of gentamicin sulfate (Manufacturer: Invitrogen, Cat. no.: J67488.AD), and 10 ng/ml of animal-free recombinant human epidermal growth factor (EGF) (Manufacturer: PeproTech, Cat. no.: AF-100-15), followed by cultivation in an incubator at 37° C. with 5% CO₂. Medium change was performed every two to three days. When the cultured cells reached approximately 80% to 90% confluence, trypsin-ethylenediaminetetraacetic acid (EDTA) (Manufacturer: Gibco, Cat. no.: 25200-072) was added to detach the cultured cells for subsequent passaging.

### Experimental Procedures

First, the CSKs prepared in section 1 of “Experimental Materials” of this example were divided into 2 groups, including an experimental group and a comparative group. The CSKs of each group were seeded at a concentration of 8×10⁶ cells/well in a 10-cm Petri dish containing 10 mL of DMEM/F12 medium. Subsequently, the CSKs of the experimental group were treated with 1 mM of vitamin C (Manufacturer: Sigma-Aldrich, Cat. no.: A2218), whereas the CSKs of the comparative group received no treatment. Next, each group of the CSKs was cultured in an incubator (37° C., 5% CO₂) for 48 hours, followed by collecting the resultant cell culture supernatant containing exosomes from the 10-cm Petri dish.

Thereafter, the cell culture supernatant of each group was subjected to an isolation treatment using an ExoQuick-TC kit (Manufacturer: System Biosciences) in accordance with the manufacturer's instructions, so as to obtain the exosome. Briefly, the cell culture supernatant of each group was subjected to a centrifugation treatment at 3000×g for 15 minutes, so as to obtain a first supernatant. Subsequently, the first supernatant was mixed with an ExoQuick-TC reagent and then left undisturbed at 4° C. for 12 hours, so as to form a mixture. Afterwards, the mixture was subjected to the following procedures conducted in sequence: performing a centrifugation treatment at 1500×g for 30 minutes; removing the resultant second supernatant; conducting another centrifugation treatment at 1500×g for 5 minutes; and removing the resultant third supernatant performed, so as to collect a pellet thus formed. The pellets of the experimental group and the comparative group were then suspended in 1× phosphate-buffered saline (PBS), so as to obtain an exosome suspension of the experimental group (abbreviated as exosome suspension 1) and an exosome suspension of the comparative group (abbreviated as exosome suspension 2), respectively.

Next, a respective one of the exosome suspensions 1 and 2 was subjected to determination of vitamin C concentration using a vitamin C colorimetric assay kit (Manufacturer: IMT Formosa New Materials Company, Ltd., Cat. no.: 300) in accordance with the manufacturer's instructions. The results showed that the vitamin C was not detected in both the exosomes suspensions 1 and 2. These results demonstrate that not only the exosome suspension 2, which was produced by the CSKs that had not been treated with the vitamin C, was free of endogenous vitamin C, but also the exosome suspension 1, which was produced by the CSKs that had been treated with the vitamin C, was also free of vitamin C.

### Example 2. Evaluation of the Effect of Exosome of the Present Disclosure on Promoting Proliferation of Corneal Endothelial Cells

### Experimental Materials

### 1. Source and Cultivation of Corneal Endothelial Cell Line B4G12

Corneal endothelial cell line B4G12 (purchased from Creative Bioarray) was grown in a T25 flask containing human endothelial serum free medium (Manufacturer: Invitrogen, Cat. no.: 11111-044) supplemented with 2% FBS and 10 ng/ml of recombinant human fibroblast growth factor (FGF)-2 (Manufacturer: PeproTech, Cat. no.: 100-18B), followed by cultivation in an incubator at 37° C. with 5% CO₂. Medium change was performed every two to three days. When the cultured cells reached approximately 80% to 90% confluence, the trypsin-EDTA was added to detach the cultured cells for subsequent passaging.

### Experimental Procedures

First, the B4G12 cells prepared in section 1 of “Experimental Materials” of this example were divided into 7 groups, including a blank control group, three experimental groups (i.e., experimental groups 1 to 3), and three comparative groups (i.e., comparative groups 1 to 3). The B4G12 cells of each group were seeded at a concentration of 1×10⁵ cells/well into a respective well of 6-well culture plates containing 1 mL of human endothelial serum free medium. Next, the B4G12 cells of each of the experimental groups 1 to 3 and the comparative groups 1 to 3 were treated with a suitable amount of the respective one of the exosome suspensions 1 and 2 prepared in Example 1, thereby having a final exosome concentration as shown in Table 1 below. In addition, the B4G12 cells of the blank control group received no treatment. Thereafter, the B4G12 cells of each group were then cultured in an incubator at 37° C. for 48 hours.

TABLE 1

| | | Final exosome |
|---|---|---|
| Group | Testing reagent | concentration (mg/mL) |
| Blank control group | — | — |
| Experimental group 1 | Exosome suspension 1 | 0.01 |
| Experimental group 2 | Exosome suspension 1 | 0.05 |
| Experimental group 3 | Exosome suspension 1 | 0.1 |
| Comparative group 1 | Exosome suspension 2 | 0.01 |
| Comparative group 2 | Exosome suspension 2 | 0.05 |
| Comparative group 3 | Exosome suspension 2 | 0.1 |

Afterwards, the liquid in each well was removed and the cell culture of each group was then subjected to trypan blue staining using a staining protocol well-known to those skilled in the art. Thereafter, each group of the stained cells was observed and photographed using an inverted microscope, followed by conducting cell counting using a hemocytometer, so as to determine cell count of each group.

### Results

The FIGURE shows the cell count determined in each group. As shown in the FIGURE, compared to the blank control group, the cell count determined in each of the comparative groups 1 to 3 showed no substantial difference, whereas the cell count determined in each of the experimental groups 1 to 3 showed a significant increase, and was significantly higher than that determined in the corresponding comparative group treated with same final exosome concentration of the exosome suspension. These results indicate that the exosome prepared by the method of the present disclosure can exhibit an excellent effect on promoting proliferation of the B4G12 cells in a dose-dependent manner.

Summarizing the above test results, it is clear that the exosome prepared by the method of the present disclosure can effectively promote proliferation of corneal endothelial cells, and hence can improve wound healing in a corneal endothelium by promoting repair and regeneration of the same.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, FIGURE, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, the one or more features may be singled out and practiced alone without the another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for preparing an exosome, comprising:
cultivating corneal stromal keratocytes (CSKs) in a culture medium containing vitamin C, so as to obtain a cell culture of the CSKs containing the exosome; and
subjecting the cell culture to an isolation treatment, so as to obtain the exosome.

2. An exosome, which is prepared by the method as claimed in claim 1.

3. A method for promoting proliferation of corneal endothelial cells, comprising administering to a subject in need thereof a composition including the exosome as claimed in claim 2.

4. The method as claimed in claim 3, wherein the composition is a pharmaceutical composition.

5. The method as claimed in claim 4, wherein the pharmaceutical composition is administered by a route selected from the group consisting of intraocular administration and topical ophthalmic administration.

6. A method for improving wound healing in a corneal endothelium, comprising administering to a subject in need thereof a composition including the exosome as claimed in claim 2.

7. The method as claimed in claim 6, wherein the composition is a pharmaceutical composition.

8. The method as claimed in claim 7, wherein the pharmaceutical composition is administered by a route selected from the group consisting of intraocular administration and topical ophthalmic administration.
